(19) United States
(12) Patent Application Publication
(10) Pub. No.: US 2026/0088148 A1
(43) Pub. Date: Mar. 26, 2026

(54) ARTIFICIAL-INTELLIGENCE-BASED PRESCRIPTION PRIOR AUTHORIZATION VALIDATION

(71) Applicant: Express Scripts Strategic Development, Inc., St. Louis, MO (US)

(72) Inventors: Christopher R. Markson, Hawthorne, NJ (US); Pritesh J. Shah, Paramus, NJ (US); Abbai Manyam, Montville, NJ (US); Urvashi Patel, Windermere, FL (US); Akash Dwivedi, Chicago, IL (US)

(21) Appl. No.: 19/334,095

(22) Filed: Sep. 19, 2025

Related U.S. Application Data
(60) Provisional application No. 63/696,997, filed on Sep. 20, 2024.
(60) Provisional application No. 63/720,625, filed on Nov. 14, 2024.

(51) Int. Cl. G16H 20/10; G06V 10/75; G06V 30/19; G06V 30/30; G06V 30/416; G06V 30/418; G06V 30/42; G16H 10/60

(52) U.S. Cl. CPC G16H 20/10; G06V 10/751; G06V 30/19013; G06V 30/30; G06V 30/416; G06V 30/418; G06V 30/42; G16H 10/60

(57) ABSTRACT

A computerized method for analyzing prior authorization documentation includes receiving a first set of information including a set of pages and determining a set of relevant pages. The method includes determining a set of document types associated with the set of relevant pages and performing optical character recognition. The method includes prompting a machine learning model to extract a set of request data. The method includes prompting the machine learning model with a set of prompts associated with a set of authorization criteria to determine a set of authorization data. The method includes, in response to a determination that the set of authorization data has met the set of authorization criteria, approving an authorization request. The method includes, in response to a determination that set of authorization data has not met the set of authorization criteria, rejecting the authorization request.

### CROSS-REFERENCE TO RELATED APPLICATIONS

This application claims the benefit of U.S. Prov. App. No. 63/720,625 filed Nov. 14, 2024 (Attorney Docket No. ESRX-472PV2) and U.S. Prov. App. No. 63/696,997 filed Sep. 20, 2024 (Attorney Docket No. ESRX-472PRV). This application is related to U.S. application Ser. No. 18/442,259 filed Feb. 15, 2024 (Attorney Docket No. ESRX-454US1) and U.S. application Ser. No. 18/791,539 filed Aug. 1, 2024 (Attorney Docket No. ESRX-466US1). The entire disclosures of the above applications are incorporated by reference.

### FIELD

The present disclosure relates to pharmaceutical order processing and more particularly to pharmaceutical order preauthorization.

### BACKGROUND

Many prescription products or services require prior authorization documentation from prescribers before a prescription can be filled. Prior authorization documentation is not standardized and essential information is frequently mixed with extraneous data across many pages and/or files. As the number of prescriptions for products and/or services that require preauthorization increases, a solution is required to simplify the prior authorization documentation review process.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A computerized method for analyzing prior authorization documentation includes receiving a first set of information including a set of pages. The method includes determining a set of relevant pages. The method includes determining a set of document types associated with the set of relevant pages. The method includes performing optical character recognition on the set of relevant pages to produce a first set of digitized pages. The method includes performing, based on the first set of digitized pages, optical character recognition on the set of relevant pages to produce a second set of digitized pages. The method includes based on the set of document types, selecting a set of prompts. The method includes prompting a machine learning model with the set of prompts to extract a set of request data from the second set of digitized pages. The method includes, based on the set of request data, determining a set of authorization criteria. The method includes prompting the machine learning model with a set of prompts associated with the set of authorization criteria to determine a set of authorization data from the second set of digitized pages. The method includes determining, via the machine learning model, whether the set of authorization data meets the set of authorization criteria. The method includes, in response to a determination that the set of authorization data has met the set of authorization criteria, approving an authorization request. The method includes, in response to a determination that the set of authorization data has not met the set of authorization criteria, rejecting the authorization request.

In other features, determining the set of document types associated with the set of relevant pages includes, for each respective page of the set of relevant pages, determining a set of information locations and comparing the set of information locations to a set of document templates.

In other features, a set of input to the machine learning model includes the set of relevant pages, the first set of digitized pages, and a set of confidence levels associated with the first set of digitized pages.

In other features, output of the machine learning model includes a set of markdown data that indicates a location of a set of information.

In other features, the set of request data includes at least one of a user associated with the set of request data, a prescriber associated with the set of request data, a medication associated with the set of request data, a signature associated with the prescriber, or a set of urgency data that includes at least one date.

In other features, the set of authorization criteria is based on the medication associated with the set of request data.

In other features, the method includes validating the set of authorization data by performing at least one of comparing the prescriber with a set of authorized prescribers, comparing a dosage associated with the medication to an expected dosage range, determining whether a first variable of the set of authorization data is within a specified range based on a type of the first variable, determining whether the first variable is above a threshold value based on the type of the first variable, determining whether the first variable is below a threshold value based on the type of the first variable, or determining whether the first variable is in a specified format based on the type of the first variable.

In other features, the method includes, in response to a determination that the second set of digitized pages does not include information associated with the set of authorization criteria, transmitting a request for additional information.

A computer system for analyzing prior authorization documentation includes memory hardware configured to store instructions and processor hardware configured to execute the instructions stored by the memory hardware. The instructions include receiving a first set of information including a set of pages. The instructions include determining a set of relevant pages. The instructions include determining a set of document types associated with the set of relevant pages. The instructions include performing optical character recognition on the set of relevant pages to produce a first set of digitized pages. The instructions include performing, based on the first set of digitized pages, optical character recognition on the set of relevant pages to produce a second set of digitized pages. The instructions include based on the set of document types, selecting a set of prompts. The instructions include prompting a machine learning model with the set of prompts to extract a set of request data from the second set of digitized pages. The instructions include, based on the set of request data, determining a set of authorization criteria. The instructions include prompting the machine learning model with a set of prompts associated with the set of authorization criteria to determine a set of authorization data from the second set of digitized pages. The instructions include determining, via the machine learning model, whether the set of authorization data meets the set of authorization criteria. The instructions include, in response to a determination that the set of authorization data has met the set of authorization criteria, approving an authorization request. The instructions include, in response to a determination that the set of authorization data has not met the set of authorization criteria, rejecting the authorization request.

In other features, determining the set of document types associated with the set of relevant pages includes, for each respective page of the set of relevant pages: determining a set of information locations and comparing the set of information locations to a set of document templates.

In other features, a set of input to the machine learning model includes the set of relevant pages, the first set of digitized pages, and a set of confidence levels associated with the first set of digitized pages.

In other features, output of the machine learning model includes a set of markdown data that indicates a location of a set of information.

In other features, the set of request data includes at least one of a user associated with the set of request data, a prescriber associated with the set of request data, a medication associated with the set of request data, a signature associated with the prescriber, or a set of urgency data that includes at least one date.

In other features, the set of authorization criteria is based on the medication associated with the set of request data.

A non-transitory computer-readable medium includes processor-executable instructions. The instructions include receiving a first set of information including a set of pages. The instructions include determining a set of relevant pages. The instructions include determining a set of document types associated with the set of relevant pages. The instructions include performing optical character recognition on the set of relevant pages to produce a first set of digitized pages. The instructions include performing, based on the first set of digitized pages, optical character recognition on the set of relevant pages to produce a second set of digitized pages. The instructions include based on the set of document types, selecting a set of prompts. The instructions include prompting a machine learning model with the set of prompts to extract a set of request data from the second set of digitized pages. The instructions include, based on the set of request data, determining a set of authorization criteria. The instructions include prompting the machine learning model with a set of prompts associated with the set of authorization criteria to determine a set of authorization data from the second set of digitized pages. The instructions include determining, via the machine learning model, whether the set of authorization data meets the set of authorization criteria. The instructions include, in response to a determination that the set of authorization data has met the set of authorization criteria, approving an authorization request. The instructions include, in response to a determination that the set of authorization data has not met the set of authorization criteria, rejecting the authorization request.

In other features, determining the set of document types associated with the set of relevant pages includes, for each respective page of the set of relevant pages: determining a set of information locations and comparing the set of information locations to a set of document templates.

In other features, a set of input to the machine learning model includes the set of relevant pages, the first set of digitized pages, and a set of confidence levels associated with the first set of digitized pages.

In other features, output of the machine learning model includes a set of markdown data that indicates a location of a set of information.

In other features, the set of request data includes at least one of a user associated with the set of request data, a prescriber associated with the set of request data, a medication associated with the set of request data, a signature associated with the prescriber, or a set of urgency data that includes at least one date.

In other features, the set of authorization criteria is based on the medication associated with the set of request data.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 is a functional block diagram of an example system for processing prior authorization documentation.

FIGS. 5A-5B are a flowchart of an example method for processing prior authorization documentation.

FIG. 6 is a flowchart of an example method for processing prior authorization documentation via a large language model (LLM).

FIG. 7 is a functional block diagram of example machine learning model training and usage.

FIG. 8 is a functional block diagram of an example neural network.

FIG. 9 is a functional block diagram of an example neural network with a hidden layer.

FIG. 10 is a functional block diagram of an example long short-term memory neural network.

FIG. 11 is a functional block diagram of an example system for processing prior authorization documentation.

FIG. 12 is a flowchart of an example method for processing prior authorization documentation.

FIG. 13 is a flowchart of an example method for processing prior authorization documentation.

In the drawings, reference numbers may be reused to identify similar and/or identical elements.

### DETAILED DESCRIPTION

### Introduction

Prior authorization is a process that determines whether a prescribed product or service (such as various prescription medications) will be covered by insurance. The prior authorization process requires that prescribers submit documentation as evidence that the prescription is necessary. This documentation is not standardized and can vary from prescriber to prescriber. In some scenarios, the documentation may include many pages and describe various aspects of a patient's medical history. The lengthy documentation creates a time-consuming review process, which includes locating specific information and verifying that the information is up to date and accurate. In some implementations, the documentation is transmitted as portable document format (PDF) files, faxes, emails, digital files, machine readable formats (e.g., only machine-readable digital formats), and/or other image files. In some scenarios, prior authorization requires review of specific authorization variables: as examples only, variables such as body mass index (BMI), A1C blood sugar values, and diagnosis codes. In some implementations, the authorization variables include additional and/or different information depending on the prescribed product or service.

The present disclosure describes parsing and analyzing prior authorization documentation via a large language model (LLM). In some implementations, prior authorization (PA) documentation is converted into text (from image files) via optical character recognition (OCR). In some implementations, the PA documentation is analyzed by OCR image by image. During the OCR process, metadata is generated that identifies where the text is found in the documentation (for example, a page number and other location data such as a horizontal and vertical coordinates). In some implementations, additional processing is performed via a natural language processing (NLP) model to determine whether the data is relevant to the authorization variables. In some implementations, data that is not relevant to the authorization variables is not transmitted to the LLM for analysis. In various embodiments, the analysis is performed one or more dedicated machines.

In some implementations, the LLM analyzes the PA documentation to determine whether the authorization variables are present. In some implementations, the LLM calculates some or all of the authorization variables from other data present in the documentation (for example, calculating BMI from height and weight). In some implementations, the LLM filters out data beyond a threshold age or data that is not the most recent version of a value (for example, a 30-day-old BMI value may be chosen over a 90-day-old BMI value). In some implementations, the LLM uses one or more LLM connector functions (or modules). LLM connector functions perform functions that LLMs are not optimized for (such as performing arithmetic or reviewing dates). In some implementations, authorization variables are validated (for example determining whether a value is within an expected range, determining whether a value matches a pattern that may be defined by a regular expression, etc.).

In some implementations, the analysis process begins automatically once PA documentation has been received. In some implementations, PA documentation is automatically gathered from prescribers without requiring a submission from the prescriber (for example in response to a detection of a prescription for a service or product that requires PA). In some implementations, data missing from the PA documentation is automatically gathered by requesting access to patient data or by sending a data request to the prescribing provider. In some implementations, the system automatically generates and/or transmits a prompt to resubmit the PA documentation in response to invalid or missing data (for example, age data that is outside of a defined range, such as the range 18 to 110, or age data that is inconsistent with date of birth data).

### High-Volume Pharmacy

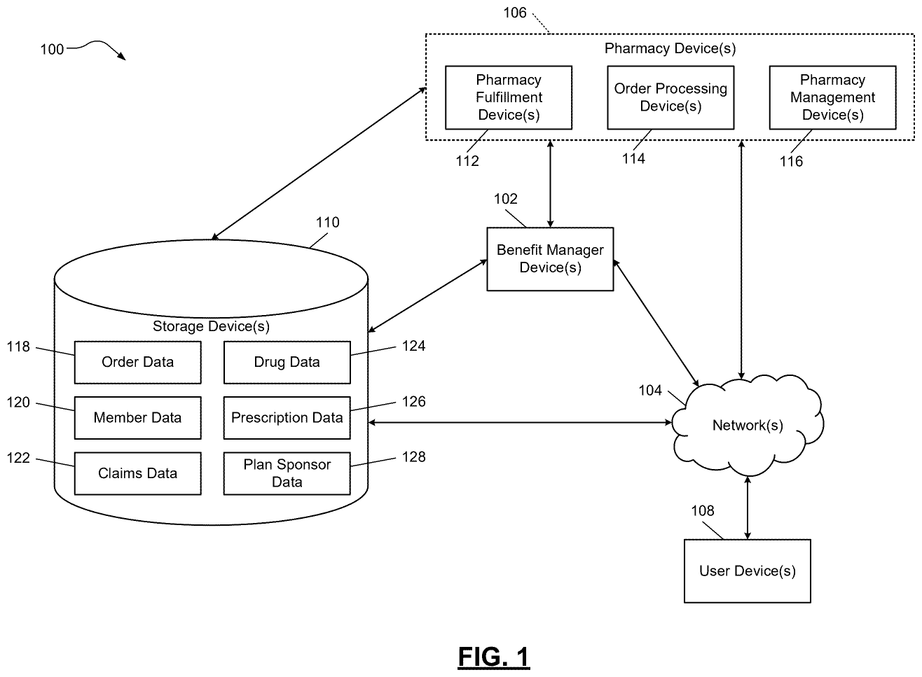

FIG. 1 is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104.

The system 100 may also include one or more user device(s) 108. A user, such as a pharmacist, patient, data analyst, health plan administrator, etc., may access the benefit manager device 102 or the pharmacy device 106 using the user device 108. The user device 108 may be a desktop computer, a laptop computer, a tablet, a smartphone, etc.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in a storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the system 100) following performance of at least some of the aforementioned operations.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Virginia.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfillment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, age, date of birth, address (including city, state, and zip code), telephone number, e-mail address, medical history, prescription drug history, etc. In various implementations, the prescription drug history may include a prior authorization claim history—including the total number of prior authorization claims, approved prior authorization claims, and denied prior authorization claims. In various implementations, the prescription drug history may include previously filled claims for the member, including a date of each filled claim, a dosage of each filled claim, the drug type for each filled claim, a prescriber associated with each filled claim, and whether the drug associated with each claim is on a formulary (e.g., a list of covered medication).

In various implementations, the medical history may include whether and/or how well each member adhered to one or more specific therapies. The member data 120 may also include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. In various implementations, the member data 120 may include an eligibility period for each member. For example, the eligibility period may include how long each member is eligible for coverage under the sponsored plan. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally or alternatively, the claims data 122 may include claims that have been de-identified (that is, associated with a unique identifier but not with a particular, identifiable member). In various implementations, the claims data 122 may include a percentage of prior authorization cases for each prescriber that have been denied, and a percentage of prior authorization cases for each prescriber that have been approved.

The drug data 124 may include drug name (e.g., technical name and/or common name), other names by which the drug is known, active ingredients, an image of the drug (such as in pill form), etc. The drug data 124 may include information associated with a single medication or multiple medications. For example, the drug data 124 may include a numerical identifier for each drug, such as the U.S. Food and Drug Administration's (FDA) National Drug Code (NDC) for each drug.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

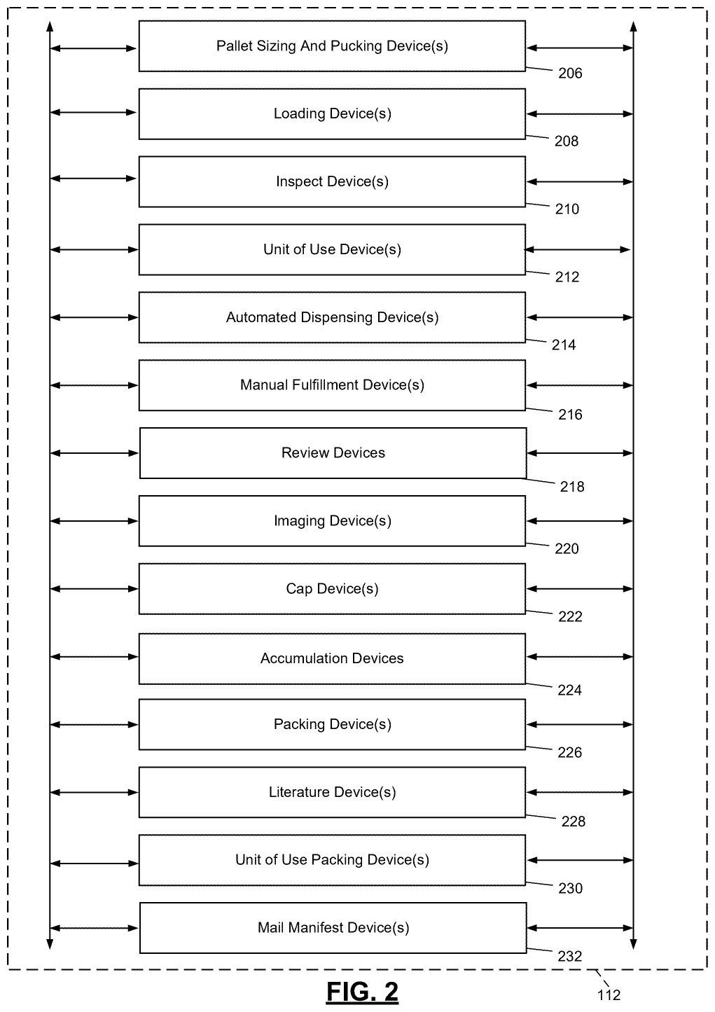

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

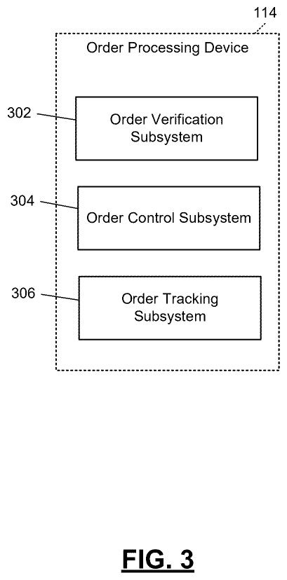

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may be comprised of order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

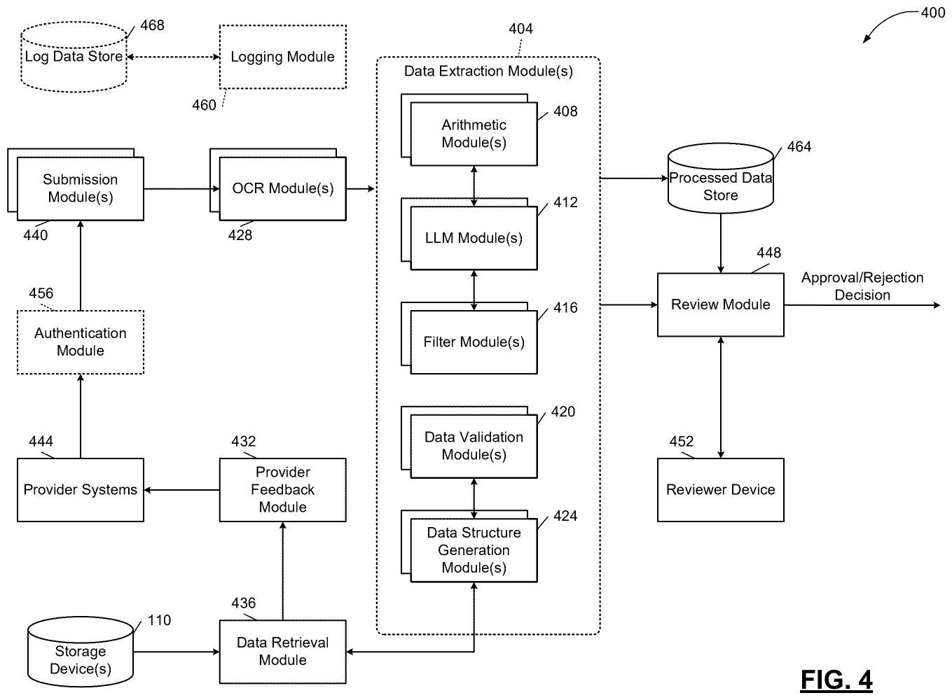

### FIG. 4

FIG. 4 is a functional block diagram of an example system 400 for processing prior authorization documentation. Submission module 440 receives prior authorization (PA) documentation from provider systems 444. In some implementations, submission module 440 includes data storage to hold PA documentation until the documentation is ready for processing. In some implementations, PA documentation is received via email, fax, web portal, or other means of digital file transfer. In some implementations, provider systems 444 is a personal computer, mobile device, automated program, and/or server(s).

In some implementations, submission module 440 is multi-threaded and runs asynchronously. For example, in some implementations, submission module 440 queues submissions (that is, delays sending submissions to OCR module 428) until a threshold level of submissions have been received so that the submissions can be processed in batches for greater efficiency. In various implementations, one or more other modules, such as OCR module 428 and/or data extraction module 404 (including one or more of its sub-modules), are multi-threaded and run asynchronously.

In some implementations, OCR module 428 and data extraction module 404 store data in a queue before the data is analyzed via OCR module 42 or LLM module 412. In some implementations, submission module 440, OCR module 428, and data extraction module 404 run as microservices. In various implementations, the microservices do not run continuously and instead run only when new submissions need analysis and/or when a queue of data has reached a threshold size or quantity. In various implementations, the microservices may scale up with volume, so that multiple instances of any high-utilization microservice may be invoked to handle the volume.

In some implementations, the PA documentation includes image files (such as JPEG, PNG, and/or TIFF) or other non-text files that must be converted into to text. PA documentation is transmitted by submission module 440 to OCR module 428. OCR module 428 performs optical character recognition (OCR) on the PA documentation and generates text data elements. OCR module 428 also generates metadata which identifies which image, page, or file within the PA documentation is the source of a respective text data elements. In some implementations, the metadata describes the location within an image, page, or file the text data element is located. In some implementations, OCR module 428 includes a natural language processing (NLP) model to determine which text data elements are related to the PA criteria (in other words, which data is extraneous and which data must be analyzed for PA approval). In some implementations, OCR module 428 filters out data that is not related to the PA criteria (for example financial data and/or address data). In some implementations, filtering is done on an element by element basis. In some implementations, filtering is done on a page by page, image by image, or file by file basis. In some implementations, data that does not require OCR processing is submitted to submission module 440; submission module 440 then sends the data directly to data extraction module 404 instead of to OCR module 428.

In some implementations, system 400 includes multiple instances of submission module 440, OCR module 428, and/or data extraction module 404 (and its sub-modules, such as LLM module 412, arithmetic module 408, filter module 416, data validation module 420, and/or data structure generation module 424). As an example, system 400 includes multiple instances of submission module 440 in order to receive, send, and/or analyze multiple batches of data at once. As another example, instances may exist for different formats of PA documentation (such as images, audio, text, PDF, email, fax, etc.). For example, instances may be spun and spun down based on the quantity of documentation requiring analysis (such as an instance per threshold file size, quantity of pages, quantity of images, quantity of text, etc.). In some implementations, the number of instances dynamically increases or decreases based on need.

Once the PA documentation has been translated into text, filtered, and associated with metadata by OCR module 428, it is transmitted to data extraction module 404. Data extraction module 404 includes LLM module 412, arithmetic module 408, filter module 416, data validation module 420, and data structure generation module 424. LLM module 412 reviews text data elements to determine if they are related to the authorization criteria and what the values of the text data elements are (for example, if the PA authorization criteria requires a specific BMI value, LLM module 412 determines whether a text data element is a BMI value, what the BMI value is, and/or if the BMI value is within the required range).

In some implementations, the authorization criteria are dynamically adjustable (for example, based on detected data attributes) and/or user selectable. For example, the authorization criteria may include BMI, A1C blood sugar values, and/or ICD 10 codes. In some implementations, a different instance of LLM module 412 is used to extract data based on the type of authorization criteria that is specified and/or detected. In some implementations, the LLMs of different instances of LLM module 412 are trained using different data and/or techniques to optimize the LLM to extract different types of data. In some implementations, the different instances of LLM module 412 use different prompts to the LLM to optimize the analysis performed by the LLM. In some implementations, the prompts are based on the type of authorization criteria, the data type of the PA documentation, and/or other characteristics such as data age or data quantity. In some implementations, the prompts are automatically adjusted based on user input. In some implementations, the various authorization criteria are weighted (for example, by their importance) by the LLM based on a user selection.

In some implementations, LLM module 412 is not optimized for certain types of analysis (such as performing arithmetic and analyzing dates). In some implementations, LLM module 412 uses arithmetic module 408 to calculate values associated with the authorization criteria. For example, if height and weight data are present in the text data elements transmitted to data extraction module 404, BMI can be calculated. In some implementations, LLM module communicates with filter module 416 to determine whether data is associated with a date, and/or whether the date is within a threshold (for example the last year, 6 months, 3 months, 30 days, etc.) In some implementations, the PA data includes multiple values associated with an authorization criterion and LLM module 412 communicates with filter module 416 to determine which value to use. In some implementations, OCR module 428 also filters data based on date before transmitting the text data elements to data extraction module 404.

Data validation module 420 determines whether text data elements associated with the authorization criteria are valid (for example, within a specified range or above a threshold value). In some implementations, data validation module 420 determines whether all required data is present. In some implementations, data structure generation module 424 saves text data elements associated with the authorization criteria with the metadata generated by OCR module 428, and data validation warnings generated by data validation module 420. In some implementations, data structure generation module 424 saves the data in a specific data structure, such as JSON, XML, or other data structure.

In some implementations, data structure generation module 424 communicates with data validation module to determine whether text data elements are invalid. In some implementations, data structure generation module 424 communicates with data retrieval module 436 to retrieve missing data from storage device 110 or request missing (and/or invalid) data from the providers. Provider feedback module 432 automatically generates and sends prompts to resubmit missing and/or invalid data to providers. In some implementations, provider feedback module 432 indicates whether data was automatically retrieved from storage devices 110. In some implementations, provider feedback module 432 automatically generates and transmits an indication that all data was found and stored via data structure generation module 424.

Once the analysis of data extraction module 404 is complete, the data structure generated by data structure generation module 424 is transmitted to review module 448. The data structure and the processed data are stored in processed data store 464. Review module 448 generates a report of the data. In some implementations, the data is displayed with a warning indication if a data value is invalid and/or missing. In some implementations, the data value is displayed with the associated metadata. In some implementations, in response to user input, the original source data (for example the image or PDF is displayed with the individual values so that the user of reviewer device 452 can confirm the data). In some implementations, the user can correct or update the data found by data extraction module 404 and the correction is reintroduced to LLM module 412 as additional training. The report is displayed on reviewer device 452. The approval or rejection decision is then transmitted by review module 448 based on user input received at reviewer device 452. In some implementations, the various modules and components of system 400 communicate via an application programming interface (API).

In some implementations, system 400 includes authentication module 456, which ensures that only authenticated entities can interact with system 400. In various implementations, authentication module 456 also manages sensitive information (such as credentials) securely. In some implementations, logging module 460 connects to one or more other modules and components of system 400 and records and stores various logs in log data store 468.

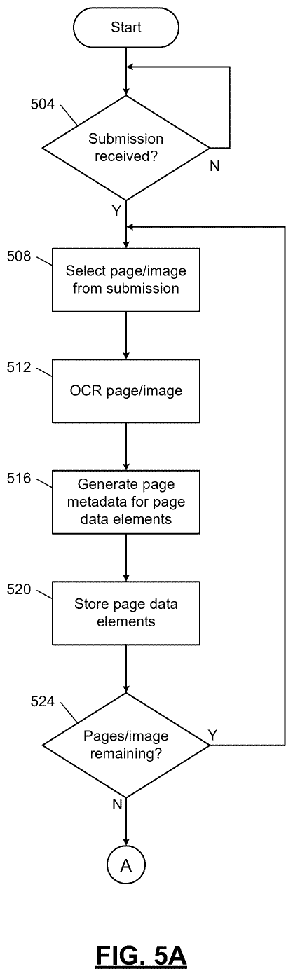

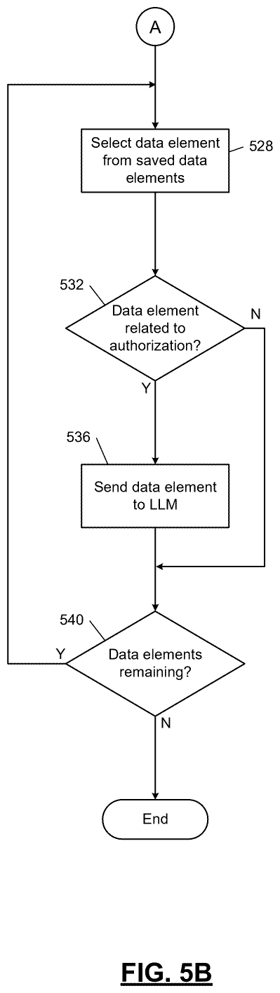

### FIGS. 5A-5B

FIGS. 5A-5B are a flowchart of an example method for processing prior authorization documentation prior to analysis by an LLM. Control begins at 504 and determines whether a submission has been received. If no submission has been received, control remains at 504. If a submission has been received, control continues to 508. In some implementations, a submission includes one or more files of various types such as PDF, images, faxes, and/or emails. In some implementations, the one or more files include one or more pages. At 508, control selects a page, image, and/or file from the submission. At 512, control performs optical character recognition (OCR) on the page, image, and/or file. In some implementations, the file is a text file and OCR is not performed. At 516, control generates metadata for the text data elements generated via OCR. In some implementations, the metadata includes location data that indicates which file, image, and/or page the text data element was generated from. In some implementations, the metadata includes location data that indicates the specific location within an image, file, and/or page data that the text data element was generated from (for example, one or more coordinates indicating which area and/or pixels the data was generated from). At 520, control saves the text data elements generated via OCR. At 524, control determines whether any pages, images, and/or files remain that have not yet been analyzed via OCR. If yes, control returns to 508. If no pages, images, and/or files remain, control transfers to 528.

At 528, control selects a first text data element from the saved elements. At 532, control determines if the data element is related to (or contains any of) the PA authorization criteria. In some implementations, unrelated data includes data outside of a date range or date threshold and/or data that is not pertinent to the PA determination, such as financial information. If the data is related to the authorization criteria, control continues to 536 and transmits the data to the LLM. If the data is not related, control transfers to 540. At 540, control determines whether there are additional data elements that require analysis. If there are additional elements remaining, control transfers to 528. If there are no remaining data elements, control ends.

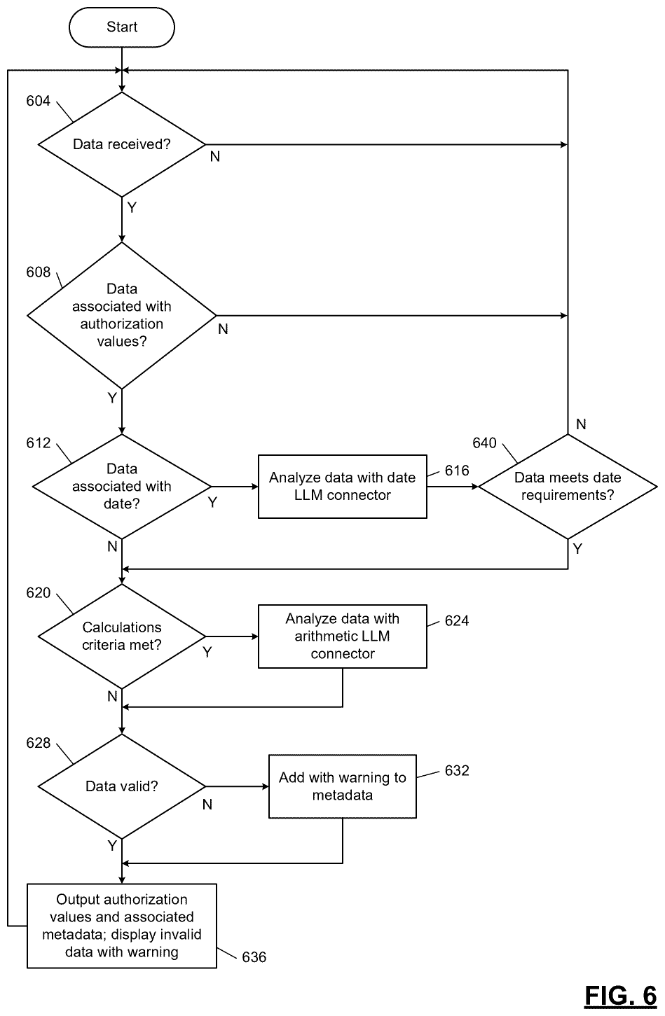

### FIG. 6

FIG. 6 is a flowchart of an example method for processing prior authorization documentation via a large language model (LLM). FIG. 6 is an example of the types of analysis performed by the LLM. In some implementations, the LLM performs the steps described below in a different order, omits some or all steps entirely, or makes similar determinations in different ways. In some implementations, the LLM determines values related to the PA criteria based on pattern recognition without performing the steps described below. In some implementations, the LLM analyzes each data element individually. In some implementations, the LLM analyzes some or all data elements as a group. In some implementations, the LLM includes (or communicates with) one or more LLM connectors that assist the LLM with various analysis processes. In some implementations, LLM connectors are modules within the LLM that perform specific functions. In some implementations, the LLM connectors are outside of the LLM and data is passed between the LLM and the LLM connectors.

Control begins at 604 and determines whether data has been received. If no data has been received, control remains at 604. If data has been received, control continues to 608. At 608, control determines whether the received data is associated with an authorization value. If the data is not associated with an authorization value, control returns to 604. If the data is associated with an authorization value, control continues to 612. In some implementations, control continues to 612 if at least a portion of the data is associated with one or more of the authorization values. At 612, control determines whether the data is associated with a date (or dates) such as an alpha-numerical indication of a day, month, week, and/or year. If yes, control transfers to 616 and the data is analyzed with an LLM connector optimized for analyzing dates. If no, control continues to 620. After 616, control continues to 640. At 640, control determines whether the data meets the date requirements (for example, whether the data is the most recent version of the data, whether data is newer than a date threshold, or within a date range). If the data does not meet the date requirements, control returns to 604. If the data meets the date requirements, control transfers to 620. In some implementations, if the data does not meet the date requirements, control continues to 620 (for example, if the data that does not meet the date requirements is part of a larger set of received data that otherwise meets the date requirements). In some implementations, if data does not meet the date requirements, that subset of data is ignored and control continues to 620.

At 620, control determines if a set of calculation criteria have been met (for example if one or more of the authorization values can be calculated from available data). In some implementations, the authorization values are calculated with related values when authorization values are already present in the data so that a comparison and/or validation can be performed between the calculated and the received values. If one or more of the calculation criteria have been met, control transfers to 624. If no calculation criteria are met, control transfers to 628. At 624, control analyzes the data with an arithmetic LLM connector and completes arithmetic as needed and control continues to 628.

At 628, control determines whether the data is valid. In some implementations, control is assisted at 628 by one or more LLM connectors. In some implementations, valid data is within a specified range for a specific authorization value type, is above a threshold value, is below a threshold value, has been found, is in a specific format based on the authorization value type, and/or other criteria. If the data is not valid, control transfers to 632 and adds a warning to the metadata associated with the data. If the data is valid, control transfers to 636. At 636, control outputs the authorization values and the associated metadata. In some implementations, the authorization values are displayed with any associated validation warnings, metadata identifying the origin location of the data, a representation of the origin location and/or file, and/or other data. After 636, control returns to 604.

### Machine Learning

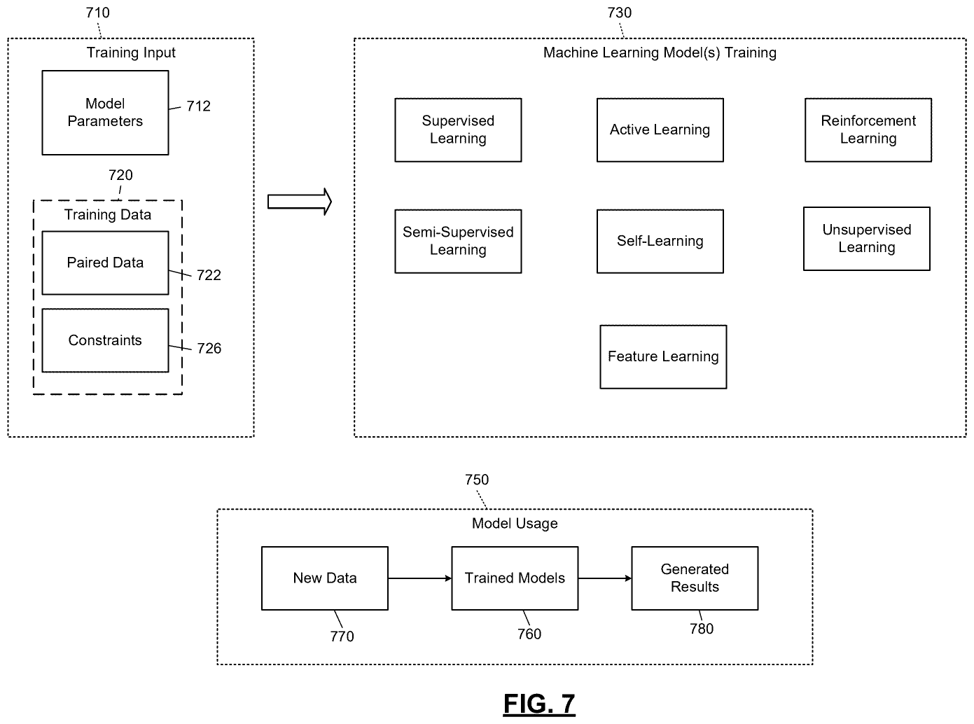

FIG. 7 is a block diagram of an example service that may be deployed above. Training input 710 includes model parameters 712 and training data 720, which may include paired training data sets 722 (e.g., input-output training pairs) and constraints 726. Model parameters 712 represents storing and/or providing the parameters or coefficients of corresponding ones of machine learning models. During training, the parameters 712 are adapted based on the input-output training pairs of the training data sets 722. After the parameters 712 are adapted (after training), the parameters are used in 750 by trained models 760 to implement the trained machine learning models on a new set of data 770.

Training data 720 optionally includes constraints 726 which may define the constraints of a given member's information features. The paired training data sets 722 optionally include sets of input-output pairs, such as pairs of a plurality of member preferences and features of entities associated with providers. Some components of training input 710 may be stored separately at a different off-site facility or facilities than other components.

Machine learning model(s) training 730 trains one or more machine learning techniques based on the sets of input-output pairs of paired training data sets 722. For example, the training 730 may train the machine learning (ML) model parameters 712 by minimizing a loss function based on one or more ground-truth data. The training 730 may include supervised learning, semi-supervised learning, active learning, self-learning, feature learning, reinforcement learning, and unsupervised learning.

The ML models can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, etc. In some implementations, the ML model is a transformer-based model.

Particularly, a first ML model of the ML models can be applied to a training batch of member preferences to estimate or generate a prediction of provider choice for a particular member. In some implementations, a derivative of a loss function is computed based on a comparison of an estimate with ground truth entities, and parameters of the first ML model are updated based on the computed derivative of the loss function. The result of minimizing the loss function for multiple sets of training data trains, adapts, or optimizes the model parameters 712 of the corresponding first ML model. In this way, the first ML model is trained to establish a relationship between member data and member selections.

After the machine learning models are trained, new data 770, including one or more sets of features for members, are received and/or derived from a document being accessed from the storage device 110. The first trained machine learning model may be applied to the new data 770 to generate results 780 (such as a prediction).

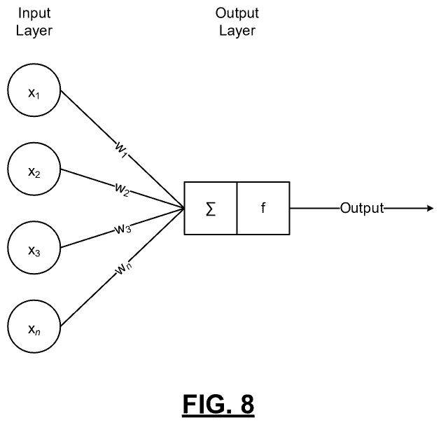

FIG. 8 is a graphical representation of an example neural network with no hidden layers for implementing a machine learning module. In machine learning, a neural network—or an artificial neural network—is a network or circuit of artificial neurons or nodes having at least an input layer and an output layer. In various implementations, neural networks may also have one or more hidden layers. Neural networks may be used in deep learning applications to allow computer systems to solve artificial intelligence problems—such as problems in predictive modeling, pattern recognition, and dynamic control systems.

FIG. 8 shows a neural network without any hidden layers. The neural network of FIG. 8 may also be referred to as a single-layer perceptron. The neural network of FIG. 8 is shown with an input layer including n nodes, labeled x₁, x₂, x₃, and xₙ. While only four nodes are illustrated in FIG. 8, the input layer may have any number of nodes. In various implementations, each node may represent any numerical value. For example, each node may represent a numerical value in a range of between 0 and 1. So, for example, the nodes of the input layer could be expressed in interval notation as: x₁∈[0, 1], x₂∈[0, 1], x₃∈[0, 1], and xₙ∈[0, 1]. In various implementations, the input variables to a neural network may be expressed as a vector i having n dimensions. In the example of FIG. 8, input vector i may be represented by equation (1) below:

$$i = 〈 {x}_{1} , {x}_{2} , {x}_{3} , {x}_{n} 〉 . \qquad ( 1 )$$

Each of the nodes may be multiplied by a weight-represented by w₁, w₂, w₃, and wₙ in FIG. 8—before being fed into a node in the next layer. In FIG. 8, because there are no hidden layers, the next layer is the output layer. For simplicity of illustration, only a single node is shown in the output layer of FIG. 8. However, the output layer may include any number of nodes.

At the node in the next layer, the inputs of the node are summed. Thus, because the inputs of the node in the output layer of FIG. 8 are the numerical value of each of the nodes of the previous layer multiplied by a weight, the summation Σ may be represented by equation (2) below:

$$\sum = {x}_{1} {w}_{1} + {x}_{2} {w}_{2} + {x}_{3} {w}_{3} + {x}_{n} {w}_{n} . \qquad ( 2 )$$

In various implementations, a bias b may be added to the nodes x of the previous layer after they have been multiplied by a weight w. For example, if biases b are added, then summation Σ may be represented by equation (3) below:

$$\sum = ( {x}_{1} {w}_{1} + {b}_{1} ) + ( {x}_{2} {w}_{2} + {b}_{2} ) + ( {x}_{3} {w}_{3} + {b}_{3} ) + ( {x}_{n} {w}_{n} + {b}_{n} ) \qquad ( 3 )$$

The summation Σ may then be fed into an activation function ƒ. The activation function ƒ may be any mathematical function suitable for calculating an output for the node. Example activation functions ƒ may include linear or non-linear functions, step functions such as the Heaviside step function, derivative or differential functions, monotonic functions, sigmoid or logistic activation functions, rectified linear unit (ReLU) functions, and/or leaky ReLU functions. The output of the function ƒ is then the output of the node. In a neural network with no hidden layers—such as the single-layer perceptron shown in FIG. 8—the output of the nodes in the output layer are the output variables or output vector of the neural network.

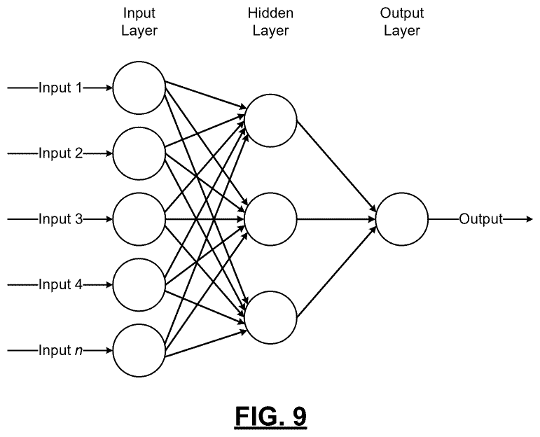

FIG. 9 is a graphical representation of an example neural network with one hidden layer for implementing the machine learning module. As illustrated in FIG. 9, the neural network may one or more intermediate layers—referred to as hidden layers—between the input layer and the output layer. The neural network of FIG. 9 may be referred to as a multilayer perceptron. Each node of a hidden layer may be connected to one or more nodes of the previous layer and receive inputs from the connected nodes of the previous layer—such as the value of the node of the previous layer multiplied by a weight (xₙwₙ) or the value of the node of the previous layer multiplied by a weight with a bias added (xₙwₙ+bₙ). Each node of the hidden layer may then function in a manner analogous to the node of the output layer of FIG. 8 by summing the inputs, feeding the summed inputs into an activation function, and feeding the output of the activation function into one or more nodes of the next layer. Similarly, the nodes of the output layer function in a manner analogous to the node of the output layer of FIG. 8. For example, the nodes of the output layer may receive the outputs of the nodes of the previous layer (multiplied by a weight and/or with a bias added as desired) as inputs, sum the received inputs, feed the summed inputs to an activation function, and output the result of the activation function as an output of the neural network.

In various implementations, the neural network may have any number of hidden layers. In various implementations, each node of a previous layer may be connected to any number of nodes of a next layer. For example, as shown in FIG. 9, each node of the previous layer may be connected to each node of the next layer. Such a neural network may be referred to as a fully-connected neural network. In various implementations, each layer of the neural network may have any number of nodes. In various implementations, a neural network with no hidden layers may function as a linear classifier and be suitable for representing linearly separable decisions or functions. In various implementations, neural networks with one hidden layer may be suitable for performing continuous mapping from one finite space to another. In various implementations, neural networks with two hidden layers may be suitable for approximating any smooth mapping to any level of accuracy.

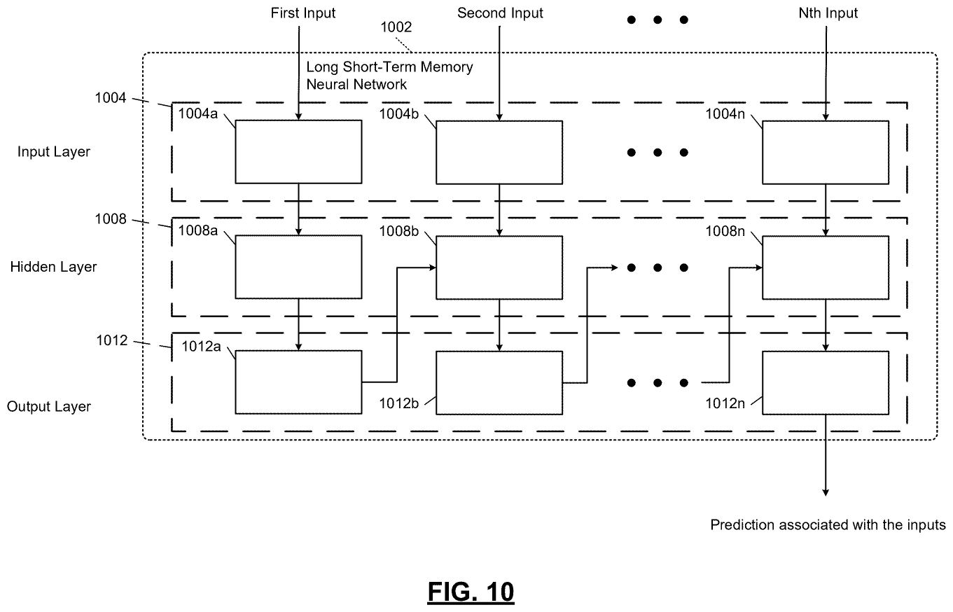

FIG. 10 is a functional block diagram of an example neural network 1002 such as an artificial neural network (ANN) that can be used to produce a predictive model. In some implementations, the neural network 1002 is a generative pre-trained transformer. In some implementations, the neural network 1002 can be a long short-term memory (LSTM) neural network. In some implementations, the neural network 1002 can be a recurrent neural network (RNN). In various embodiments, the RNN is a specific form of an ANN. The example neural network 1002 may be used to implement the machine learning as described herein, and various implementations may use other types of machine learning networks. The neural network 1002 includes an input layer 1004, a hidden layer 1008, and an output layer 1012. The input layer 1004 includes inputs 1004a, 1004b . . . 1004n. In various embodiments, the input layer 1004 includes a multi-head self-attention mechanism to allow the model to focus on different parts of the input text or input vector simultaneously, capturing various contextual relationships. The hidden layer 1008 includes neurons 1008a, 1008b . . . 1008n, which are interconnected by electrical synapses. In various embodiments, each neuron includes local processing circuitry and a local memory electrically connected to the local processing circuitry. The output layer 1012 includes outputs 1012a, 1012b . . . 1012n.

Each neuron of the hidden layer 1008 receives an input from the input layer 1004 and outputs a value to the corresponding output in the output layer 1012. For example, the neuron 1008a receives an input from the input 1004a and outputs a value to the output 1012a. Each neuron, other than the neuron 1008a, also receives an output of a previous neuron as an input. For example, the neuron 1008b receives inputs from the input 1004b and the output 1012a. In this way the output of each neuron is fed forward to the next neuron in the hidden layer 1008. The last output 1012n in the output layer 1012 outputs a probability associated with the inputs 1004a-1004n. Although the input layer 1004, the hidden layer 1008, and the output layer 1012 are depicted as each including three elements, each layer may contain any number of elements. Neurons can include one or more adjustable parameters, weights, rules, criteria, or the like.

In various implementations, each layer of the neural network 1002 must include the same number of elements as each of the other layers of the neural network 1002. For example, training features may be processed to create the inputs 1004a-1004n.

The inputs 1004a-1004n can include data features (binary, vectors, factors or the like) stored in the storage device 110. The features can be provided to neurons 1008a-1008n for analysis and connections between the known facts. The neurons 1008a-1008n, upon finding connections, provides the potential connections as outputs to the output layer 1012.

In some examples, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 1004a is connected to each of neurons 1008a, 1008b . . . 1008n.

In various embodiments, the system uses an ANN to identify or detect anomalies, and reduce errors generated in machine processing (such as in the data contained within machine readable files, documents and the like). In various embodiments, the ANN is a specially trained neural network operating as a generative pre-trained transformer model. In various embodiments, the ANN processes the entire input sequence in parallel rather than sequentially. In various embodiments, the ANN generates predictions about how well the data in the document files match the required inputs for the ultimate task. In various embodiments, the ANN determines whether inputs to the ultimate task are missing, contain errors or are anomalies. The use of specially trained ANNs to detect anomalies and errors produces improvements over traditional methods of detecting anomalies, including more accurate detection of anomalies and errors and faster processing. The present disclosure further provides methods for training an ANN that lead to faster training times and a more accurate model for detecting anomalies and errors.

An initial model (such as an RNN or ANN) can be built in a secure environment using health data relating to patients. The initial model can then be refined based on feedback with a computing system that also is in a secure environment. The health data—such as the patient name, drug name, dosing data, and other prescription information—remains within a secure computing environment and not communicated out to a public database or subject to a third-party artificial intelligence. The secure computing system mitigates the risk of working with protected health data and other types of high-risk data, such as personal identifying information or state protected data. In an example, the secure computing system is a mainframe computer with limited connection to external systems. In an example, the computing system is a private cloud environment that provides high-performance, secure, and flexible computing environments enabling the analysis of sensitive datasets governed by federal privacy laws, proprietary access agreements, or confidentiality requirements. A private cloud environment can provide creation of any combination of network, CPU, RAM, and storage components into resource groups that can be used to build multi-tenant, multi-site infrastructure as a service.

In various implementations, processing and storage for the system (such as the example system shown in FIG. 4) is split between local (on-premises) and cloud (off-site) environments. The assignment of responsibilities may be performed by system designers based on workload. For example, processing-heavy tasks, such as OCR and NLP, may be performed in the cloud environment, with other tasks being performed in the local environment. In various implementations, the cloud environment includes multiple cloud environments, which may be hosted by the same or different parties. As an example, the architecture may be capable of handling more than 1 million transactions per year, with most transactions involving more than 10 pages of OCR; as a further example, the architecture may be capable of handling 2.5 million transactions per year, with many transactions involving more than 40 pages of OCR.

In various implementations, the architecture may dynamically adjust, such as by delegating some tasks to the cloud environment based on loading of the local system. As a specific example, some OCR may be performed in the local system, but when the amount of OCR is higher or the capacity of the local system is lower, some or all of the OCR responsibilities may be offloaded to the cloud environment.

The neural networks (such as RNNs or ANNs) are a type of machine learning model used to perform a wide variety of complex tasks, including image recognition, speech recognition, pattern recognition, and detection of anomalies and errors. In various embodiments, anomalies and errors are an indication of errors and missing required data in machine readable files (for example, images, unstructured document files, structured document files, and the like). The required data is needed for a further identified task associated with a specific application. The same document files may be associated with multiple identified tasks.

In various embodiments, a neural network is an algorithm that learns from training data. A neural network can be formed through software, hardware, or a combination of software and hardware. The structure of an example neural network has a series of layers, each comprising one or more neurons arranged in one or more neuron arrays. In an example embodiment, a neuron may comprise a register, a microprocessor, and at least one input. Each neuron produces an output, or activation, based on an activation function that uses the outputs of the previous layer and a set of weights as inputs. Each neuron in a neuron array may be connected to another neuron via a synaptic circuit. A synaptic circuit may include a memory for storing a synaptic weight. An example neural network may be a Deep Neural Network (DNN) with an input layer, an output layer, and a plurality of fully connected hidden layers. Neural networks are particularly useful in anomaly detection because they can effectively extract features in linear and nonlinear relationships. In some embodiments, the neural network is implemented by an application-specific integrated circuit (ASIC) or graphical processing units. ASICs may be specially customized for a specific artificial intelligence application and provide superior computing capabilities and reduced electricity consumption compared to traditional computer processing units.

In some embodiments, training data is generated by receiving continuous data at a computer and using the computer to discretize the continuous data. In some embodiments, the continuous data may be received remotely over a network. The continuous data may be historical data, which the neural network can use to learn patterns to identify or detect potential anomalies and errors. Continuous data is measured and can have any number of possible values. Machine learning models may benefit from being trained with discrete data rather than continuous data.

Discrete data can be counted and has a limited number of values. For example, discrete data may include historical medical records that are missing data for an ultimate processing task (such as prior authorization). This can be determined by a further subsequent medical record file being received and added to a patient file stored in a database that is subsequently accessed before the ultimate processing task is completed. Any type of discretization method may be used to convert continuous data to discrete data, including binning, clustering, and numerical discretization. The neural network is then trained using training techniques to generate a trained neural network (e.g., a predictive model) which can be used to detect anomalies and errors.

The trained neural network performs the processes described herein and monitors incoming data sets to detect anomalies and errors for an ultimate processing task. The ultimate processing task can have at least one same data requirement from the machine-readable files relative to a further ultimate processing task. The ultimate processing task can have at least one different data requirement from a further ultimate processing task. If the trained neural network detects one or more anomalies or errors (which can include missing data field for the ultimate processing task), it additionally analyzes the detected anomalies to generate anomaly data which can be output to a user, automatically request further documents that may contain the missing data, and/or used to re-train the neural network. For example, the anomaly data may explain the type of anomaly or a cause of the anomaly.

In various embodiments, an application specific integrated circuit (ASIC) for an ANN executes the processes described herein and includes a plurality of neurons organized in an array (in other words, multiple inputs and multiple layers). Each neuron comprises a register (in other words, a memory), processor circuitry, at least one input, and a plurality of synaptic circuits. Each synaptic circuit includes a memory for storing a synaptic weight for that neuron. Each neuron is connected to at least one other neuron via one of the plurality of synaptic circuits. In various embodiments, the ASIC operates the artificial neural network to detect various data fields across multiple discrete machine readable files in inputs to identify data required for an ultimate processing task (such as identification of data across multiple input files, medical claim review, prior authorization review, and the like).

The method trains, by a computer, the ANN based on input data and a selected training algorithm to generate a trained ANN, wherein the selected training algorithm includes a first algorithm and a second algorithm. In an example, the first algorithm is a Markov decision process. The method detects one or more present values defined by the ultimate task and, if present, one or more missing values required by the ultimate task using the artificial neural network. The artificial neural network can further determine that the at least one missing value must be supplied by a further document file and requesting the further document or requesting the missing data field from another source. The artificial neural network can detect a source address to request the missing data field from data it finds in the current machine-readable documents.

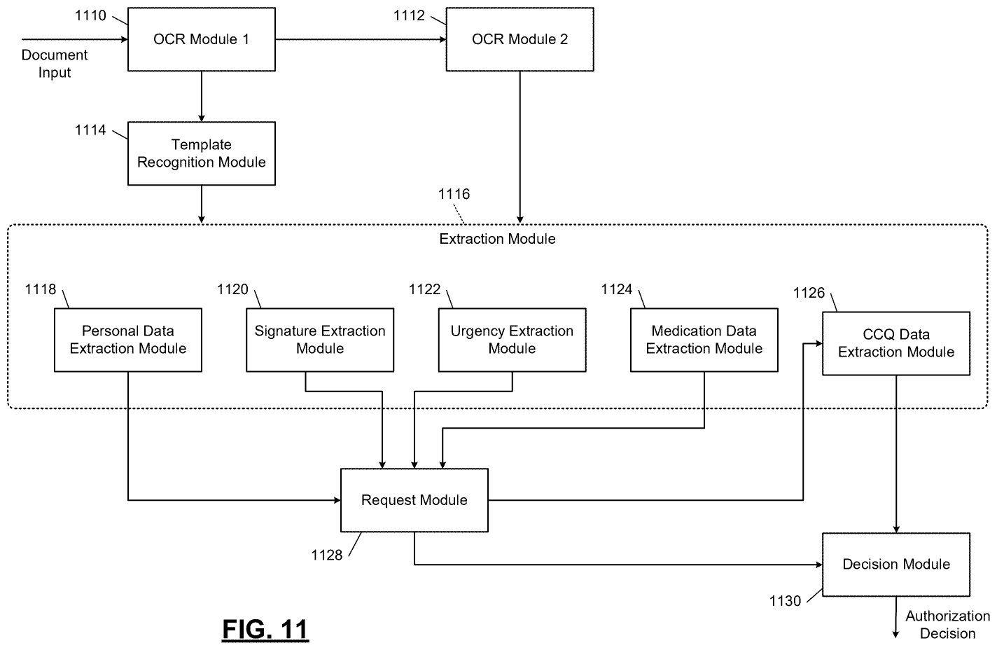

### FIG. 11

FIG. 11 is a functional block diagram of an example system for processing prior authorization documentation. Document input (for example, an email, a scanned document, a faxed document, etc.) is received at OCR module 1 (1110), which identifies relevant pages of the received document. In some scenarios received documents include hundreds of pages. Identifying which pages contain relevant information can save time and processing power. In various embodiments, relevant pages are determined based on whether the pages contain information that is generally related to prior authorization questions (such as whether the page contains patient or prescriber information, signatures, etc.). In various embodiments, OCR module 1 (1110) includes one or more LLMs which determine the relevancy of information on a page.

The output (such as digitized pages) of OCR module 1 (1110) is passed to OCR module 2 (1112). In various embodiments, OCR module 2 (1112) receives the OCR output of OCR module 1 (1110), the confidence level associated with the output, and the undigitized relevant pages. In various embodiments, OCR module 2 performs a secondary OCR and compares the results from both OCR modules. In various embodiments, OCR module 2 (1112) selects the OCR digitization with the highest confidence rating. In various embodiments, OCR module 2 (1112) generates a location markdown of the relevant pages, which indicate the physical location of information on a page. In various embodiments, OCR module 2 (1112) includes one or more LLMs to determine the relevance of information and/or identify text. In various embodiments, the LLM determines relevancy based on one or more prompts.

The output of OCR module 1 (1110) is also received at template recognition module 1114, which compares received pages with a set of templates to determine the type of document. For example, template recognition module 1114 determines whether a received page consistent with Form A or Form B. In various embodiments, template identification includes using Euclidean distance comparisons and/or a grid search, identifying columns, identifying columns, etc. In various embodiments, potential matching templates are assigned a confidence or matching score, and the most likely candidate is selected. In various embodiments, the confidence score is based on a how close a particular document feature is to a specific location. In various embodiments, the determination includes a polygon search (in a particular region), which helps determine whether a document matches a template when the document is skewed, shrunken, rotated, etc. By identifying a document type (in other words, whether a document matches a template) time and processing power can be saved by using document-specific extraction prompts. For example, describing the location of patient information, a signature, medication information, etc. via a document-specific prompt. In various embodiments, if a document cannot be matched with a template, a general prompt is used which instructs an extraction LLM to a generic prompt (for example, a prompt that instructs the LLM to use the mark up information to extract data from when the data is located).

The output of template recognition module 1114 and OCR module 2 (1112) are passed to extraction module 1116. In various embodiments, extraction module 1116 includes one or more sub-modules such as personal data extraction module 1118, signature extraction module 1120, urgency extraction module 1122, medication data extraction module 1124, and clinical criteria questions (CCQ) extraction module 1126.

In various embodiments, personal data extraction module 1118 includes or communicates with one or more LLMs. Based on the document identification from template recognition module 1114, personal data extraction module 1118 selects one or more predetermined prompts to extract personal data such as patient (name, age, patient medical data, etc.) and/or prescriber information from the digitized documents.

In various embodiments, signature extraction module 1120 includes or communicates with one or more LLMs. Based on the document identification from template recognition module 1114, signature extraction module 1120 selects one or more predetermined prompts to extract signatures (such as prescriber signatures approving medication) from the digitized documents.

In various embodiments, urgency extraction module 1122 includes or communicates with one or more LLMs. Based on the document identification from template recognition module 1114, urgency extraction module 1122 selects one or more predetermined prompts to extract date information from the digitized documents.

In various embodiments, medication data extraction module 1124 includes or communicates with one or more LLMs. Based on the document identification from template recognition module 1114, medication data extraction module 1124 selects one or more predetermined prompts to extract which medication has been requested from the digitized documents.

Request module 1128 combines the extracted personal data, the extracted signature data, the extracted urgency data, and the extracted medication data to determine what medication is being requested. In various embodiments, different types of requests are received by the same system, therefore it is necessary to differentiate the different types of requests.

Based on the request generated by request module 1128, CCQ data extraction module 1126 determines the relevant clinical criteria questions (CCQ) for the medication. For example, for medication A, a patient's age, blood pressure, and weight may be relevant to the prior authorization determine. For example, for medication B, a patient's BMI may be relevant. In various embodiments, CCQ data extraction module 1126 includes or communicates with one or more LLMs. Based on the document identification from template recognition module 1114, CCQ data extraction module 1126 selects one or more predetermined prompts to extract data relevant to the CCQs.

Based on the data from CCQ data extraction module 1126, decision module 1130 determines whether to approve or deny the prior authorization request. In various embodiments, decision module 1130 includes one or more LLMs that analyzes the CCQ data and CCQ questions. In various embodiments, decision module 1120 includes a user interface sub-module that displays the decision for human approval. In various embodiments, decision module 1130 receives the request data (both the digitized pages and the source image data) and displays the information for human review. In various embodiments, human review can replace various elements of the identified information if it is incorrect. In various embodiments, the human edits are used as LLM training to improve accuracy.

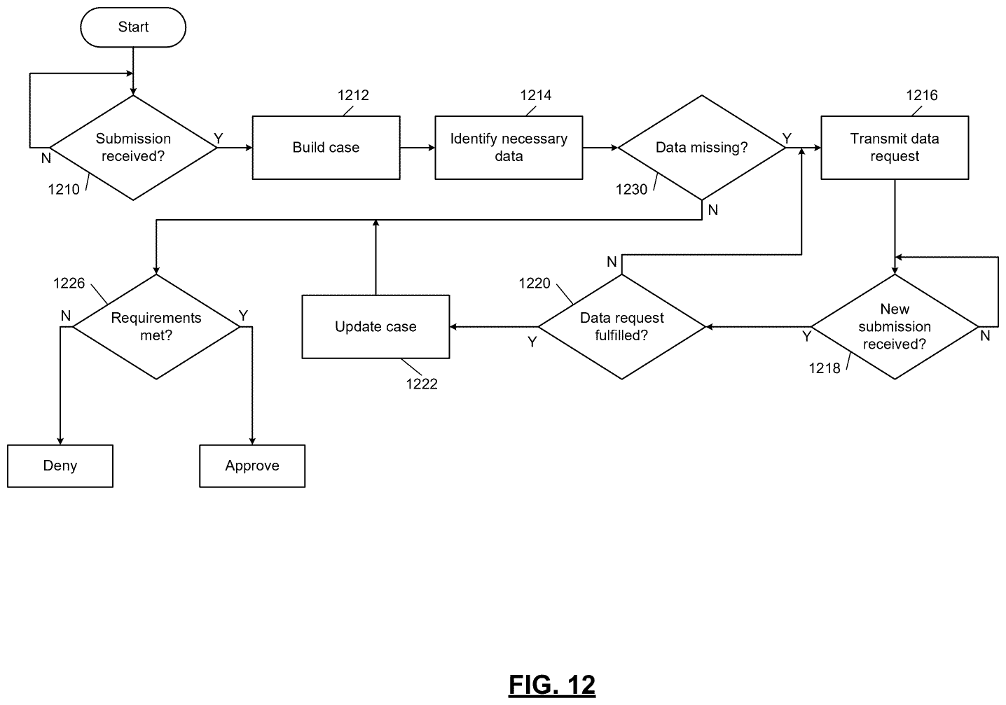

### FIG. 12

FIG. 12 is a flowchart of an example method for processing prior authorization documentation. In various embodiments, control begins at 1210 and determines whether a submission (in other words, a prior authorization request) has been received. If a submission has not been received, control remains at 1210. If a submission has been received, control transfers 1212. At 1212, control builds a case file for the submission by extracting relevant data (for example patient and medication information as described with respect to FIG. 11). At 1214, control determines which data is relevant to the request (and in various embodiments, determines what the request is). At 1230, control determines whether the request contains all data necessary to make a prior authorization determination. If all the necessary data is present, control transfers to 1226. If data is missing, control transfers to 1216.

At 1216, control transmits a data request for the missing data (for example, to the medication prescriber) and waits for a new submission. At 1218, control determines whether a new submission has been received. If no submission has been received, control remains at 1218. If a new submission has been received, control continues to 1220. At 1220, control determines whether the data request has been fulfilled (for example, does the new submission contain the requested data). If the new submission does not contain the requested data, control returns to 1216. If new submission contains the requested data, control transfers to 1222 and updates the case file. After 1222, control continues to 1226 and determines whether the prior authorization requirements have been met.

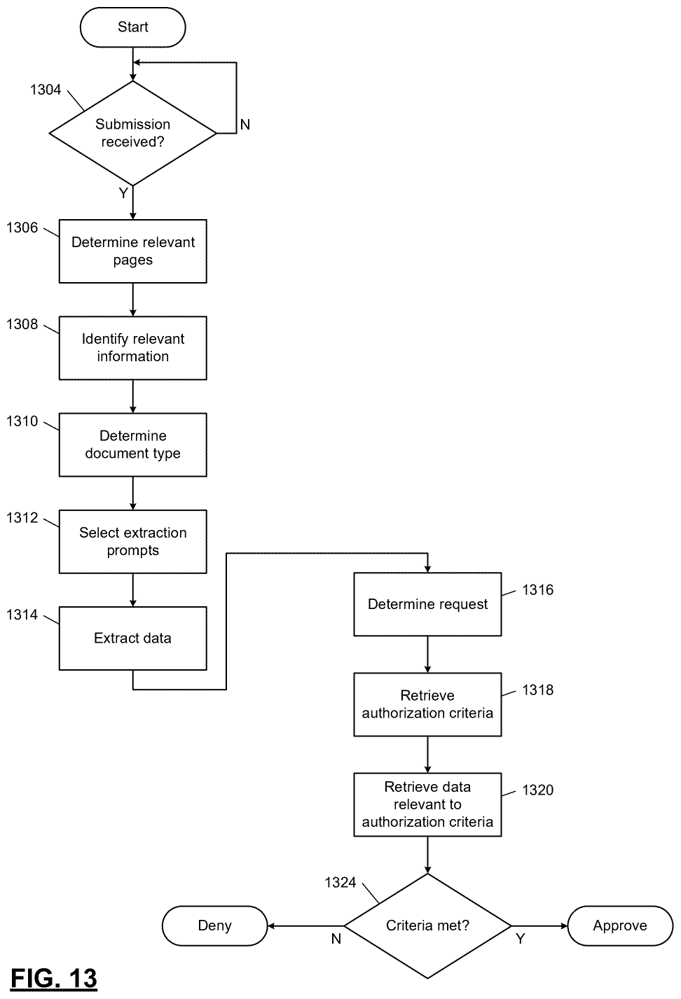

### FIG. 13

FIG. 13 is a flowchart of an example method for processing prior authorization documentation. Control begins at 1304 and determines whether a submission has been received. If a submission has not been received, control remains at 1304. If a submission has been received, control continues to 1306. At 1306, control determines the relevant pages of the submission. At 1308, control determines the relevant information on the pages. At 1310, control determines the document type of the relevant pages. At 1312, control selects data extraction prompts based on the document type. At 1314, control extracts data using the selected prompts. At 1316, based on the extracted information, control determines what is being requested. At 1318, based on the request, control determines (or retrieves) the authorization criteria for the request. At 1320, control retrieves data (from the extracted data) relevant to the authorization criteria. At 1324, control determines whether the authorization criteria have been met.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in a different order (or concurrently) without altering the principles of the present disclosure. Unless indicated otherwise, numbering or other labeling of instructions or method steps is done for convenient reference, not to indicate a fixed order.

Numerical terms, such as “first,” “second,” and “third,” may be used in the disclosure and claims as unique labels: unless the context clearly indicates otherwise, they are not used to imply a sequence or order. In other words, a “second” element could be relabeled as a “first” element without departing from the principles of the present disclosure. Further, the presence of a “second” element does not imply or require the presence of a “first” element. Similarly, the presence of a “first” element does not imply or require the presence of a “second” element.

Unless the context clearly indicates otherwise, the singular articles “a,” “an,” and “the” before a noun do not restrict the noun to a single instance. The verbs “comprise,” “include,” and “have” are inclusive and therefore specify the presence of elements without excluding the presence of one or more additional elements.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules, circuit elements, semiconductor layers, etc.) are described using various terms, including “connected,” “coupled,” “engaged,” “adjacent,” “next to,” “on top of,” “above,” “below,” and “disposed.” Unless explicitly described as being “direct,” when a relationship between first and second elements is described in the above disclosure, that relationship encompasses a direct relationship where no other intervening elements are present between the first and second elements as well as an indirect relationship where one or more intervening elements are present (either spatially or functionally) between the first and second elements.

The term “set” generally means a grouping of one or more elements. The elements of a set do not necessarily need to have any characteristics in common or otherwise belong together. However, in various implementations a “set” may, in certain circumstances, be the empty set (in other words, the set has zero elements in those circumstances). As an example, a set of search results resulting from a query may, depending on the query, be the empty set. In contexts where it is not otherwise clear, the term “non-empty set” can be used to explicitly denote exclusion of the empty set—that is, a non-empty set will always have one or more elements.

A “subset” of a first set generally includes some of the elements of the first set. In various implementations, a subset of the first set is not necessarily a proper subset: in certain circumstances, the subset may be coextensive with (equal to) the first set (in other words, the subset may include the same elements as the first set). In contexts where it is not otherwise clear, the term “proper subset” can be used to explicitly denote that a subset of the first set must exclude at least one of the elements of the first set. Further, in various implementations, the term “subset” does not necessarily exclude the empty set. As an example, consider a set of candidates that was selected based on first criteria and a subset of the set of candidates that was selected based on second criteria; if no elements of the set of candidates met the second criteria, the subset may be the empty set. In contexts where it is not otherwise clear, the term “non-empty subset” can be used to explicitly denote exclusion of the empty set.

The phrase “at least one of A, B, and C” should be construed to mean a logical (A OR B OR C), using a non-exclusive logical OR, and should not be construed to mean “at least one of A, at least one of B, and at least one of C.” The phrase “at least one of A, B, or C” should be construed to mean a logical (A OR B OR C), using a non-exclusive logical OR. The phrase “A, B, and/or C” should be construed in the same way as the phrase “at least one of A, B, and C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgments of, the information to element A.

In this application, including the definitions below, the term “module” can be replaced with the term “controller” or the term “circuit.” In this application, the term “controller” can be replaced with the term “module.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); processor hardware (shared, dedicated, or group) that executes code; memory hardware (shared, dedicated, or group) that is coupled with the processor hardware and stores code executed by the processor hardware; other suitable hardware components that provide the described functionality; or a combination of some or all of the above, such as in a system-on-chip.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2020 (also known as the WIFI wireless networking standard) and IEEE Standard 802.3-2018 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

Some or all hardware features of a module may be defined using a language for hardware description, such as IEEE Standard 1364-2005 (commonly called “Verilog”) and IEEE Standard 1076-2008 (commonly called “VHDL”). The hardware description language may be used to manufacture and/or program a hardware circuit. In some implementations, some or all features of a module may be defined by a language, such as IEEE 1666-2005 (commonly called “SystemC”), that encompasses both code, as described below, and hardware description.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

The memory hardware may also store data together with or separate from the code. Shared memory hardware encompasses a single memory device that stores some or all code from multiple modules. One example of shared memory hardware may be level 1 cache on or near a microprocessor die, which may store code from multiple modules. Another example of shared memory hardware may be persistent storage, such as a solid state drive (SSD) or magnetic hard disk drive (HDD), which may store code from multiple modules. Group memory hardware encompasses a memory device that, in combination with other memory devices, stores some or all code from one or more modules. One example of group memory hardware is a storage area network (SAN), which may store code of a particular module across multiple physical devices. Another example of group memory hardware is random access memory of each of a set of servers that, in combination, store code of a particular module. The term memory hardware is a subset of the term computer-readable medium.

The apparatuses and methods described in this application may be partially or fully implemented by a special-purpose computer created by configuring a general-purpose computer to execute one or more particular functions embodied in computer programs. Such apparatuses and methods may be described as computerized or computer-implemented apparatuses and methods. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special-purpose computer, device drivers that interact with particular devices of the special-purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

The term non-transitory computer-readable medium does not encompass transitory electrical or electromagnetic signals propagating through a medium (such as on a carrier wave). Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

1. A computerized method for analyzing prior authorization documentation comprising:
receiving a first set of information including a set of pages;
determining a set of relevant pages;
determining a set of document types associated with the set of relevant pages;
performing optical character recognition on the set of relevant pages to produce a first set of digitized pages;
performing, based on the first set of digitized pages, optical character recognition on the set of relevant pages to produce a second set of digitized pages;
based on the set of document types, selecting a set of prompts;
prompting a machine learning model with the set of prompts to extract a set of request data from the second set of digitized pages;
based on the set of request data, determining a set of authorization criteria;
prompting the machine learning model with a set of prompts associated with the set of authorization criteria to determine a set of authorization data from the second set of digitized pages;
determining, via the machine learning model, whether the set of authorization data meets the set of authorization criteria;
in response to a determination that the set of authorization data has met the set of authorization criteria, approving an authorization request; and
in response to a determination that the set of authorization data has not met the set of authorization criteria, rejecting the authorization request.

2. The computerized method of claim 1, wherein determining the set of document types associated with the set of relevant pages includes, for each respective page of the set of relevant pages:
determining a set of information locations; and
comparing the set of information locations to a set of document templates.

3. The computerized method of claim 1, wherein a set of input to the machine learning model includes:
the set of relevant pages;
the first set of digitized pages; and
a set of confidence levels associated with the first set of digitized pages.

4. The computerized method of claim 1, wherein output of the machine learning model includes a set of markdown data that indicates a location of a set of information.

5. The computerized method of claim 1, wherein the set of request data includes at least one of:
a user associated with the set of request data;
a prescriber associated with the set of request data;
a medication associated with the set of request data;
a signature associated with the prescriber; or
a set of urgency data that includes at least one date.

6. The computerized method of claim 5, wherein the set of authorization criteria is based on the medication associated with the set of request data.

7. The computerized method of claim 5, further comprising validating the set of authorization data by performing at least one of:
comparing the prescriber with a set of authorized prescribers;
comparing a dosage associated with the medication to an expected dosage range;
determining whether a first variable of the set of authorization data is within a specified range based on a type of the first variable;
determining whether the first variable is above a threshold value based on the type of the first variable;
determining whether the first variable is below a threshold value based on the type of the first variable; or
determining whether the first variable is in a specified format based on the type of the first variable.

8. The computerized method of claim 1, further comprising:
in response to a determination that the second set of digitized pages does not include information associated with the set of authorization criteria, transmitting a request for additional information.

9. A computerized system for analyzing prior authorization documentation comprising:
memory hardware configured to store instructions; and
processor hardware configured to execute the instructions stored by the memory hardware, wherein the instructions include:
receiving a first set of information including a set of pages;
determining a set of relevant pages;
determining a set of document types associated with the set of relevant pages;
performing optical character recognition on the set of relevant pages to produce a first set of digitized pages;
performing, based on the first set of digitized pages, optical character recognition on the set of relevant pages to produce a second set of digitized pages;
based on the set of document types, selecting a set of prompts;
prompting a machine learning model with the set of prompts to extract a set of request data from the second set of digitized pages;
based on the set of request data, determining a set of authorization criteria;
prompting the machine learning model with a set of prompts associated with the set of authorization criteria to determine a set of authorization data from the second set of digitized pages;
determining, via the machine learning model, whether the set of authorization data meets the set of authorization criteria;
in response to a determination that the set of authorization data has met the set of authorization criteria, approving an authorization request; and
in response to a determination that the set of authorization data has not met the set of authorization criteria, rejecting the authorization request.

10. The computerized system of claim 9, wherein determining the set of document types associated with the set of relevant pages includes, for each respective page of the set of relevant pages:
determining a set of information locations; and
comparing the set of information locations to a set of document templates.

11. The computerized system of claim 9, wherein a set of input to the machine learning model includes:
the set of relevant pages;
the first set of digitized pages; and
a set of confidence levels associated with the first set of digitized pages.

12. The computerized system of claim 9, wherein output of the machine learning model includes a set of markdown data that indicates a location of a set of information.

13. The computerized system of claim 9, wherein the set of request data includes at least one of:
a user associated with the set of request data;
a prescriber associated with the set of request data;
a medication associated with the set of request data;
a signature associated with the prescriber; or
a set of urgency data that includes at least one date.

14. The computerized system of claim 13, wherein the set of authorization criteria is based on the medication associated with the set of request data.

15. A non-transitory computer-readable medium comprising processor-executable instructions including:
receiving a first set of information including a set of pages;
determining a set of relevant pages;
determining a set of document types associated with the set of relevant pages;
performing optical character recognition on the set of relevant pages to produce a first set of digitized pages;
performing, based on the first set of digitized pages, optical character recognition on the set of relevant pages to produce a second set of digitized pages;
based on the set of document types, selecting a set of prompts;
prompting a machine learning model with the set of prompts to extract a set of request data from the second set of digitized pages;
based on the set of request data, determining a set of authorization criteria;
prompting the machine learning model with a set of prompts associated with the set of authorization criteria to determine a set of authorization data from the second set of digitized pages;
determining, via the machine learning model, whether the set of authorization data meets the set of authorization criteria;
in response to a determination that the set of authorization data has met the set of authorization criteria, approving an authorization request; and
in response to a determination that the set of authorization data has not met the set of authorization criteria, rejecting the authorization request.

16. The non-transitory computer-readable medium of claim 15, wherein determining the set of document types associated with the set of relevant pages includes, for each respective page of the set of relevant pages:
determining a set of information locations; and
comparing the set of information locations to a set of document templates.

17. The non-transitory computer-readable medium of claim 15, wherein a set of input to the machine learning model includes:
the set of relevant pages;
the first set of digitized pages; and
a set of confidence levels associated with the first set of digitized pages.

18. The non-transitory computer-readable medium of claim 15, wherein output of the machine learning model includes a set of markdown data that indicates a location of a set of information.

19. The non-transitory computer-readable medium of claim 15, wherein the set of request data includes at least one of:
a user associated with the set of request data;
a prescriber associated with the set of request data;
a medication associated with the set of request data;
a signature associated with the prescriber; or
a set of urgency data that includes at least one date.

20. The non-transitory computer-readable medium of claim 19, wherein the set of authorization criteria is based on the medication associated with the set of request data.
